(19) United States
(12) Patent Application Publication
(10) Pub. No.: US 2021/0276023 A1
(43) Pub. Date: Sep. 9, 2021

(54) ALKYL THIOETHER ETHYL HYDROXAMIC ACID BENEFICIATION REAGENT AND PREPARATION METHOD AND APPLICATION THEREOF

(71) Applicant: CENTRAL SOUTH UNIVERSITY, Changsha (CN)

(72) Inventors: Hong ZHONG, Changsha (CN); Qing SUN, Changsha (CN); Shuai WANG, Changsha (CN)

(73) Assignee: CENTRAL SOUTH UNIVERSITY, Changsha (CN)

(21) Appl. No.: 15/734,226

(22) PCT Filed: Jun. 19, 2020
(86) PCT No.: PCT/CN2020/096986

§ 371 Date: Dec. 2, 2020

(30) Foreign Application Priority Data
Sep. 4, 2019 (CN) 201910830505.3

(51) Int. Cl. B03D 1/012

(52) U.S. Cl. CPC B03D 1/012; B03D 2203/02; B03D 2201/02

(57) ABSTRACT

An alkyl thioether ethyl hydroxamic acid beneficiation reagent and a preparation method and application thereof are provided. The alkyl thioether ethyl hydroxamic acid molecules have two functional groups, a thioether group and a hydroxamate group. The beneficiation reagent is obtained by esterification of alkyl thioether acetic acid and methanol and then by hydroxamation of hydroxylamine and an alkali. The alkyl thioether ethyl hydroxamic acid beneficiation reagent can be used as a collector for mineral flotation. The preparation method is simple and has a high yield. The thioether and hydroxamate group in the molecules have a synergistic effect and can effectively improve the collection performance.

### BACKGROUND

### Technical Field

The invention belongs to the field of beneficiation reagents and specifically relates to a novel alkyl thioether ethyl hydroxamic acid beneficiation reagent and a preparation method and application thereof.

### Related Technology

Hydroxamic acid compounds are typical chelating agents with efficient selectivity for metal ions. Because molecular structures of the hydroxamic acid compounds has oxygen and nitrogen containing lone pair electrons which are close to each other, the hydroxamic acid compounds can chelate with metal ions to form stable chelates and form five-membered ring structures by combining two O atoms in carbonyl and hydroxyl groups with metal cations. With such a special structure, the hydroxamic acid compounds have been widely used in the fields of oxidized ore flotation, solvent extraction, wastewater treatment, medicine and so on.

Wang et al. reported flotation of alkyl hydroxamic acids on fine cassiterite and their solution chemistry (structural formula a, Peipei Wang, Wenqing Qin, Liuyi Ren, et al. Solution chemistry and utilization of alkyl hydroxamic acid in flotation of fine cassiterite[J]. Transactions of Nonferrous Metals Society of China, 2013, 23(6): 1789-1796). Zuo et al. have confirmed through many experimental studies that uranium fuels after reduction and extraction can well achieve separation and purification of uranium and plutonium by being treated with an organic extraction phase containing acetohydroxamic acid (structure b, Chen Zuo, Taihong Yan, Weifang Zheng, et al. Kinetics and mechanism of stripping of Np(IV) by acetohyroxamic acid using a Lewis cell[J]. Journal of Radioanalytical and Nuclear Chemistry, 2010, 283(1): 83-87). US Patent Application No. US20020143052A1 reported that aryl fatty acids and hydroxamic acid are used as histone deacetylase inhibitors for treatment of cancer, blood diseases and genetic-related metabolic diseases (structure c).

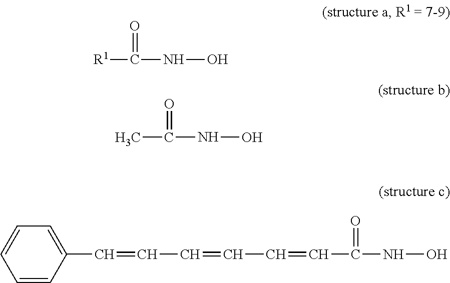

At present, hydroxamic acid collectors commonly used in the flotation industry are still dominated by hydroxamic acids with short carbon chains such as alkyl hydroxamic acid, benzohydroxamic acid and salicylhydroxamic acid. These hydroxamic acids generally have good selectivity, but low collection ability. There is no report about the use of the alkyl thioether ethyl hydroxamic acid reagent as a collector for mineral flotation at present.

### SUMMARY

An objective of the present invention is to provide an alkyl thioether ethyl hydroxamic acid beneficiation reagent with a new structure in view of the defects of existing oxide ore collectors.

Another objective of the present invention is to provide a preparation method of the alkyl thioether ethyl hydroxamic acid beneficiation reagent.

A third objective of the present invention is to provide application of the alkyl thioether ethyl hydroxamic acid beneficiation reagent as a collector which can be widely used in flotation of bauxite, tungsten ores, copper oxide ores, tin ores and other oxide ores. Compared with commonly used hydroxamic acid collectors in the industry, the alkyl thioether ethyl hydroxamic acid beneficiation reagent has better selectivity for target minerals and higher flotation efficiency.

The present invention discloses an alkyl thioether ethyl hydroxamic acid beneficiation reagent, having a structure as shown in formula I,

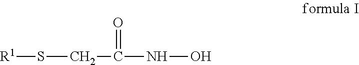

In the formula I, R¹ is a C₁-C₁₂ alkane group, a C₅-C₁₂ cycloalkyl group, a C₆-C₁₂ aromatic group or a C₁-C₁₂ alkane group substituted with at least one substituent.

In the present invention, R¹ is the C₁-C₁₂ alkane group, such as a linear alkane group or a branched alkyl group.

R¹ may also be the C₅-C₁₂ cycloalkyl group, preferably a five-membered or six-membered cycloalkane group, the cycloalkane group may have a substituent, and the substituent may be at least one of halogen and alkyl.

The C₆-C₁₂ aromatic group may be at least one of phenyl, benzyl, or a benzene ring, containing at least one of an alkane group or halogen.

The substituent of the C₁-C₁₂ alkane group substituted with at least one substituent may be at least one of phenyl, benzyl and p-tert-butyl benzyl.

Preferably, R¹ is one of methyl, ethyl, propyl, isopropyl, butyl, isobutyl, sec-butyl, tert-butyl, pentyl, isopentyl, sec-pentyl, neopentyl, cyclopentyl, n-hexyl, isohexyl, sec-hexyl, cyclohexyl, heptyl, cycloheptyl, n-octyl, isooctyl, sec-octyl, cyclooctyl, benzyl, phenyl, p-tert-butyl benzyl and dodecyl.

More preferably, R¹ is one of benzyl and dodecyl.

The present invention also provides a preparation method of the alkyl thioether ethyl hydroxamic acid beneficiation reagent, which includes the following steps:

(1) an esterification reaction: alkyl thioether acetic acid with a structure as shown in formula II and methanol are subjected to an esterification reaction under the catalysis of concentrated sulfuric acid to obtain methyl alkyl thioether acetate with a structure as shown in formula III;

(2) a hydroximation reaction: the methyl alkyl thioether acetate with the structure as shown in formula III, hydroxylamine and an alkali are subjected to a hydroxmation reaction in an aqueous solution to obtain the alkyl thioether ethyl hydroxamic acid beneficiation reagent;

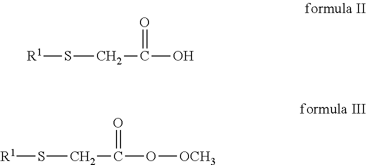

In the formulas II and III, R¹ is a C₁-C₁₂ alkane group, a C₅-C₁₂ cycloalkyl group, a C₆-C₁₂ aromatic group or a C₁-C₁₂ alkane group substituted with at least one substituent.

Preferably, in the step (1), the esterification reaction temperature is 50-100° C., the reaction time is 1-6 hours, a molar ratio of the alkyl thioether acetic acid to the methanol is 1:(1-8), a mass fraction of the concentrated sulfuric acid is 25-50 g/mol, and an addition amount is 2.5-5 g/0.1 mol alkyl thioether acetic acid.

Preferably, in the step (2), the hydroxmation reaction temperature is 10-60° C., the reaction time is 2.5-6 hours, the hydroxylamine is hydroxylamine hydrochloride or hydroxylamine sulfate, the alkali is sodium hydroxide or potassium hydroxide, a molar ratio of the methyl alkyl thioether acetate to the hydroxylamine to the alkali is 1:(1-1.5):(1-1.5), and the amount of water is 10-100 mL water/0.1 mol methyl alkyl thioether acetate.

The present invention also provides application of the alkyl thioether ethyl hydroxamic acid beneficiation reagent as a collector for flotation of metallic ores.

Preferably, the metallic ores are at least one of bauxite, tungsten ores, copper oxide ores and tin ores.

The alkyl thioether ethyl hydroxamic acid beneficiation reagent of the present invention is used as a flotation collector to efficiently recover valuable metals from metallic ores. The thioether and hydroxamate groups in the flotation collector have the effects of synergistically chelating with metal ions, enhancing the effect of the collector on metal ions on mineral surfaces and promoting efficient recovery of minerals.

In the present invention, in flotation process of the metallic ore, after pulp is prepared, the alkyl thioether ethyl hydroxamic acid collector is added, and metallic minerals are floated by the froth flotation method. Preferably, a basic process of using alkyl thioether ethyl hydroxamic acid as a collector includes: (1) flotation of metallic ores after grinding; (2) preparation of a saline solution of alkyl thioether ethyl hydroxamic acid as a flotation reagent from mixing the alkyl thioether ethyl hydroxamic acid of formula I and sodium hydroxide or potassium hydroxide in water; (3) addition of hydrochloric acid or sodium hydroxide during flotation to adjust the pulp pH to 7-9, and addition of 25-400 mg/L saline solution of alkyl thioether ethyl hydroxamic acid under weakly alkaline conditions; and (4) flotation of useful metallic minerals by the froth flotation method.

The alkyl thioether ethyl hydroxamic acid collector used in the present invention has high collection ability for bauxite, malachite, tin ores, wolframite and other minerals and can improve the flotation recovery rate of the minerals. To use the alkyl thioether ethyl hydroxamic acid of the present invention as a collector, the dosage of the alkyl thioether ethyl hydroxamic acid reagent is 25-400 mg/L, and hydrochloric acid or sodium hydroxide is added during flotation to adjust the pulp pH to 7-9. Compared with benzohydroxamic acid, the alkyl thioether ethyl hydroxamic acid can increase the flotation recovery rate of oxide ores by about 30% under weakly alkaline conditions, thereby achieving flotation separation of valuable and gangue minerals.

Compared with the existing technology, the beneficial effects of the present invention are as follows:

1. A compound containing thioether and hydroxamate groups is applied to flotation separation of non-ferrous metal minerals for the first time to realize efficient recovery of non-ferrous metal minerals.

2. The collector is a compound with a complex functional group. To be specific, it has a complex functional group of —S— and a hydroxamate group. The two functional groups have great synergistic chelation effect, good collection effect and high chelating ability for the metal ions.

3. Compared with current flotation collectors commonly used in the industry, the thioether-containing hydroxamic acid collector with the structure of the present invention has good collection performance. Compared with that of benzohydroxamic acid, the hydrophobic hydrocarbon chain of the present invention is relatively long, which can improve the hydrophobic foaming performance of the collector and the froth flotation efficiency. The non-ferrous metal recovery process is simple, efficient and feasible, and can meet the requirements of industrial application.

4. At present, thionocarbamates wastewater contains a large amount of thioglycolic acid in the industry, and the raw material of the present invention is derived from thioglycolic acid, improving the recovery of valuable substances in wastewater.

### BRIEF DESCRIPTION OF THE DRAWINGS

FIG. 1 is a nuclear magnetic resonance hydrogen spectrum of 2-(benzylthio)-acetohydroxamic acid;

FIG. 2 is a nuclear magnetic resonance carbon spectrum of 2-(benzylthio)-acetohydroxamic acid;

FIG. 3 is an infrared spectrum of 2-(benzylthio)-acetohydroxamic acid;

FIG. 4 is a nuclear magnetic resonance hydrogen spectrum of 2-(dodecylthio)-acetohydroxamic acid;

FIG. 5 is a nuclear magnetic resonance carbon spectrum of 2-(dodecylthio)-acetohydroxamic acid;

FIG. 6 shows the optimal configuration of 2-(benzylthio)-acetohydroxamic acid at the level of DFT/B3LYP6-311G(d);

FIG. 7 shows the optimal configuration of benzohydroxamic acid at the level of DFT/B3LYP6-311G(d);

FIG. 8 shows the highest occupied molecular orbital (HOMO) and lowest unoccupied molecular orbital (LUMO) of 2-(benzylthio)-acetohydroxamic acid at the level of DFT/B3LYP6-311 G(d);

FIG. 9 shows the highest occupied molecular orbital (HOMO) and lowest unoccupied molecular orbital (LUMO) of benzohydroxamic acid at the level of DFT/B3LYP6-311G(d);

FIG. 10 shows the molecular electrostatic potential of 2-(benzylthio)-acetohydroxamic acid at the level of DFT/B3LYP6-311G(d);

FIG. 11 shows the molecular electrostatic potential of benzohydroxamic acid at the level of DFT/B3LYP6-311G(d);

FIG. 12 is a schematic diagram of molecular structure and atomic numbers of benzohydroxamic acid and 2-(benzylthio)-acetohydroxamic acid;

FIG. 13 is a flowchart of a wolframite flotation process in Example 6 of the present invention.

### DETAILED DESCRIPTION

The present invention is further described with reference to the following examples, but is not limited by these examples.

### Example 1

Preparation of 2-(Benzylthio)-Acetohydroxamic Acid:

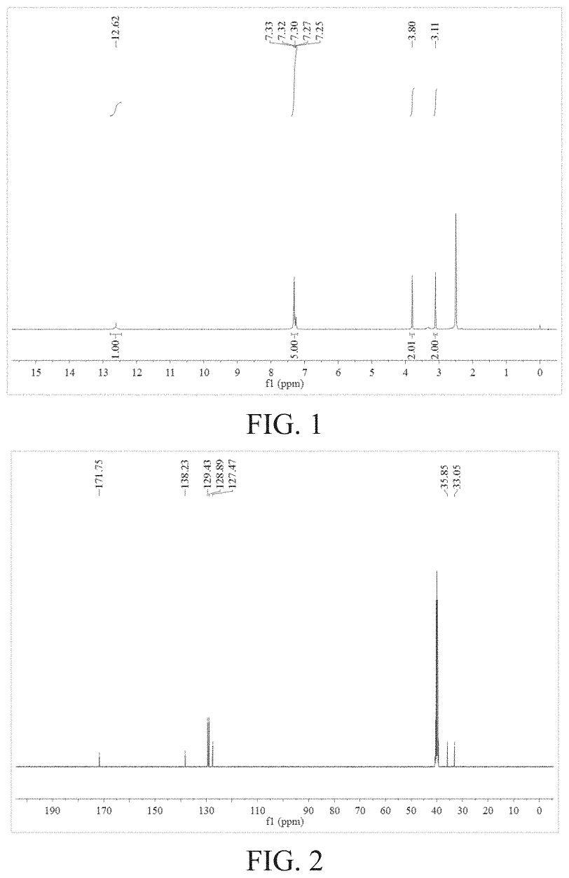

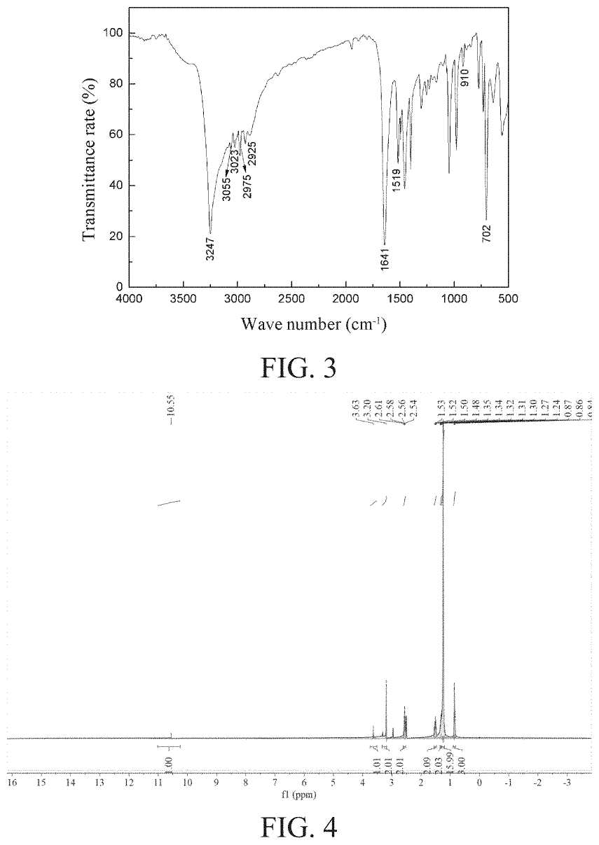

For the first step, 18.93 g of 96.15% 2-(benzylthio)acetic acid, 16.16 g of 99% methanol and 2.5 g of 98% concentrated sulfuric acid were added to a 150 mL three-neck flask. After the mixture was heated at 75° C. for 5 hours, the temperature was cooled to room temperature. Then, 4.2 g of 98.5% solid sodium bicarbonate was added, the mixture was filtered and distilled under reduced pressure to remove methanol to obtain methyl S-benzylthioglycolate. For the second step, 7.76 g of 99.5% hydroxylamine hydrochloride and 30 mL distilled water were added to a 150 mL three-neck flask. Under stirring, 8.33 g of 85.0% sodium hydroxide and 20 mL distilled water were added to the mixture in batches, under 5° C. heat. Next, methyl S-benzylthioglycolate was added dropwise to the mixture. After a reaction time of 4 h at 40° C., the mixture was acidified with sulfuric acid to obtain 16.81 g product of 2-(benzylthio)-acetohydroxamic acid with a yield of 91.86% based on 2-(benzylthio)acetic acid. 2-(benzylthio)-acetohydroxamic acid was characterized after purification, with ¹H NMR, ¹³C NMR and infrared spectrum being as shown in FIGS. 1 to 3 respectively.

TABLE 1
Analysis results of nuclear magnetic resonance hydrogen spectrum and carbon spectrum

| Compound | NMR | |
|---|---|---|
| 2-(benzylthio)-acetohydroxamic | ¹H NMR/δ | δ: 2.91(2H, —CH2—), 3.83(2H, —CH2—), |
| acid (the solvent is deuterated | | 7.25(5H, —C6H5), 8.93(1H, —NH—), |
| DMSO) | | 10.58(—OH) |
| | ¹³C NMR/δ | δ: 31.66(1C, S—CH2—), 36.13(1C, —CH2—S), |
| | | 127.41(1C, Ar—CH); 128,88(2C, 2Ar—CH); |
| | | 129.44(2C, Ar—CH); 138.47(1C, CH); |
| | | 166.41(1C, C═O). |

TABLE 2
Analysis results of infrared spectrum

| Compound | Peak shift and possible attribution |
|---|---|
| 2-(benzylthio)-acetohydroxamic | 3247 cm⁻¹ is —OH or —NH stretching vibration; 3055 and |
| acid | 302.3 cm⁻¹ are C═CH—H stretching vibration on a benzene |
| | ring; 2975 and 2925 cm⁻¹ are —CH₂— stretching vibration; |
| | 1641 cm⁻¹ is C═O stretching vibration; 1519 cm⁻¹ is C—N |
| | stretching vibration; 910 cm⁻¹ is C—S stretching vibration; |
| | 702 cm⁻¹ is benzene ring bending vibration. |

Quantum chemistry calculation results show that a hydrophobic constant ClogP value of 2-(benzylthio)-acetohydroxamic acid is 0.9626, energy values of the highest occupied molecular orbital (HOMO) and lowest unoccupied molecular orbital (LUMO) of molecules are −0.24699 and −0.03267 respectively. An energy gap between the highest occupied molecular orbital and the lowest unoccupied molecular orbital can be used as a stability index for organic compounds. The energy gap of 2-(benzylthio)-acetohydroxamic acid is 0.21432, which is close to the benzohydroxamic acid (Table 3). As a result, 2-(benzylthio)-acetohydroxamic acid has high collection ability and good selectivity and is especially suitable for flotation of copper oxide ores, bauxite, tungsten ores, tin ores and other oxide minerals.

It can be seen from Table 4 and FIG. 10 that the N—O bond length of 2-(benzylthio)-acetohydroxamic acid is similar to that of benzohydroxamic acid, but the C═O double bond is longer. It indicates that electrons of 2-(benzylthio)-acetohydroxamic acid are less distributed between the C═O double bonds, which results in weaker strength of the C═O double bonds than benzohydroxamic acid, and therefore the activity is higher. Hydroxamic acid interacts with minerals through combination of two O atoms in carbonyl and hydroxyl groups and metal cations to form a five-membered ring structure. Therefore, 2-(benzylthio)-acetohydroxamic acid is more likely to interact with metal cations. Dihedral angle data show that the dihedral angle composed of 2-(benzylthio)-acetohydroxamic acid O4-C3-N2-O1 is closer to 0 than benzohydroxamic acid, which is conducive to formation of conjugated n bonds, improving the effect of metal ions with the minerals and facilitating a more stable chelating ring after interacting with metal ions.

TABLE 3
Single-point energy, HOMO and LUMO energy values and CLogP value of the
hydroxamic acid collector at the level of DFT/B3LYP6-311G(d)

| | | | HOMO- | | |
|---|---|---|---|---|---|
| | HOMO | LUMO | LUMO | Energy E_{T} | |
| Collector | (a.u.) | (a.u.) | (a.u.) | (a.u.) | ClogP |
| Benzohydroxamic acid | −0.27071 | −0.05732 | 0.21339 | −476.24628 | 0.255 |
| 2-(benzylthio)-acetohydroxamic | −0.24699 | −0.03267 | 0.21432 | −953.10377 | 0.9626 |
| acid | | | | | |

TABLE 4
Structural parameters of the benzohydroxamic acid collector at the level of DFT/B3LYP6-311G(d)

| | r1 | r2 | r3 | α1 | α2 | τ |
|---|---|---|---|---|---|---|
| Collector | (O1-N2) | (N2-C3) | (C3-O4) | (O1-N2-C3) | (N2-C3-O4) | (O4-C3-N2-O1) |
| 2-(benzylthio)- | 1.39 | 1.34 | 1.24 | 118.59 | 120.42 | 6.54566 |
| acetohydroxamic acid | | | | | | |
| Bertzollydroxamic acid | 1.39 | 1.38 | 1.22 | 117.49 | 122.93 | −13.17814 |

### Example 2

Preparation of 2-(Benzylthio)-Acetohydroxamic Acid:

For the first step, 9.47 g of 96.15% 2-(benzylthio)acetic acid, 8.08 g of 99% methanol and 1.3 g of 98% concentrated sulfuric acid were added to a 100 mL three-neck flask. After the mixture was heated at 75° C. for 5 hours, the temperature was cooled to room temperature. Then, 2.1 g of 98.5% solid sodium bicarbonate was added, the mixture was filtered and distilled under reduced pressure to remove methanol to obtain methyl S-benzylthioglycolate. For the second step, 3.88 g of 99.5% hydroxylamine hydrochloride and 30 mL distilled water were added to a 100 mL three-neck flask. Under stirring, 6.59 g of 85.0% sodium hydroxide and 20 mL distilled water were added to the mixture in batches, under 5° C. heat. Next, methyl S-benzylthioglycolate was added dropwise to the mixture. After a reaction time of 4 h at 40° C., the mixture was acidified with sulfuric acid to obtain 8.92 g product of 2-(benzylthio)-acetohydroxamic acid with a yield of 90.56% based on 2-(benzylthio)acetic acid. Example 3

Preparation of 2-(Dodecylthio)-Acetohydroxamic Acid:

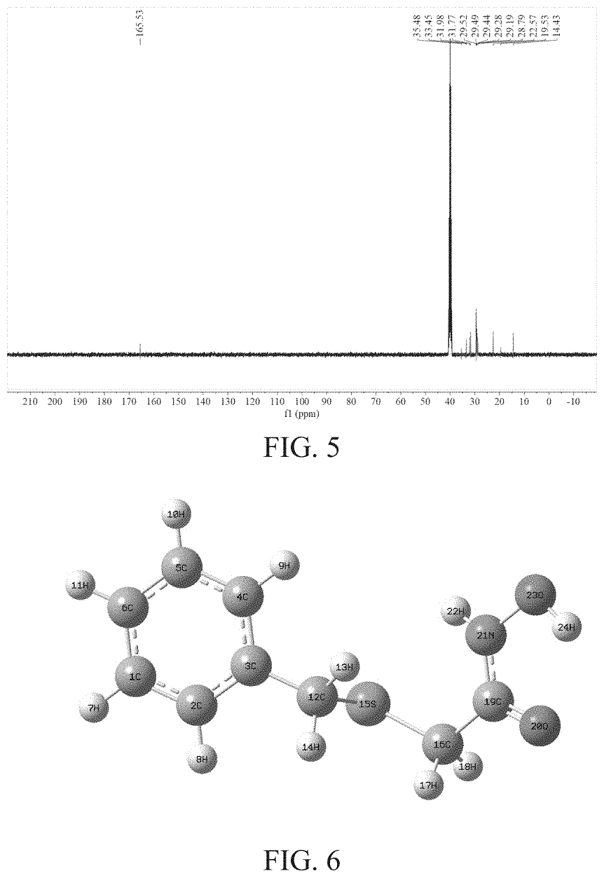

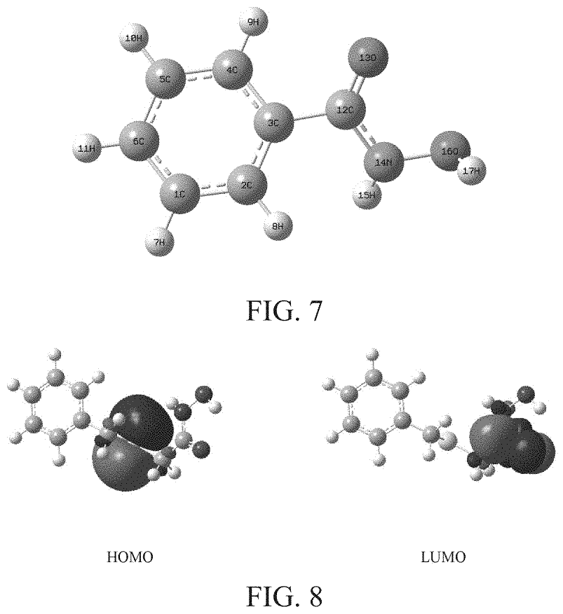

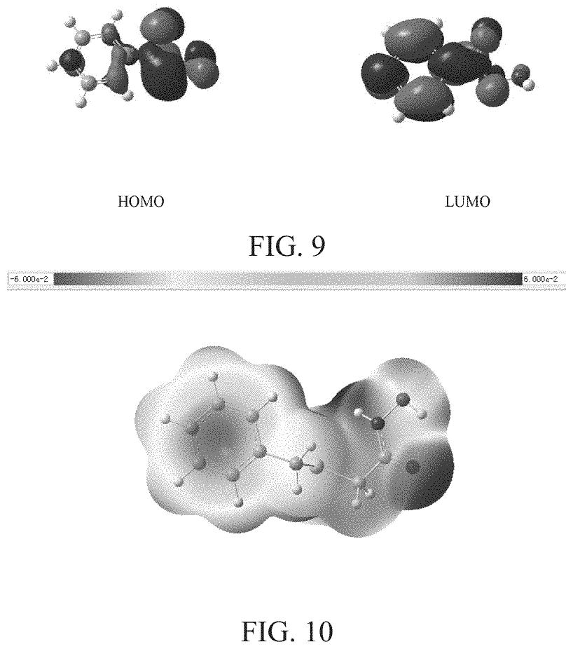

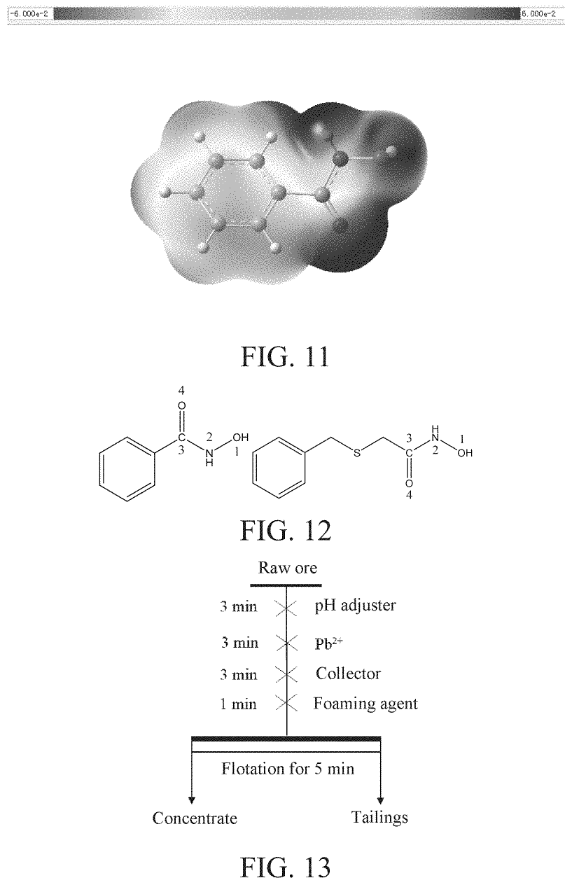

For the first step, 18.71 g of 97.30% 2-(dodecylthio)acetic acid, 16.16 g of 99%/6 methanol and 2.5 g of 98% concentrated sulfuric acid were added to a 150 mL three-neck flask. After the mixture was heated at 75° C. for 4.5 hours, the temperature was cooled to room temperature. Then, 4.2 g of 98.5% solid sodium bicarbonate was added, the mixture was filtered and distilled under reduced pressure to remove methanol to obtain methyl S-dodecylthioglycolate. For the second step, 7.76 g of 99.5% hydroxylamine hydrochloride and 30 mL distilled water were added to a 150 mL three-neck flask. Under stirring, 8.33 g of 85.0% sodium hydroxide and 20 mL distilled water were added to the mixture in batches, under 5° C. heat. Next, methyl S-dodecylthioglycolate was added dropwise to the mixture. After a reaction time of 4 h at 40° C., the mixture was acidified with sulfuric acid to obtain 17.20 g product of 2-(dodecylthio)-acetohydroxamic acid with a yield of 89.30% based on 2-(dodecylthio)acetic acid, with ¹H NMR and ¹³C NMR being as shown in FIGS. 4 to 5 respectively.

TABLE 5
Analysis results of nuclear magnetic resonance hydrogen spectrum and carbon spectrum

| Compound | NMR | |
|---|---|---|
| 2-(dodecylthio)-acetohydroxamic | ¹H NMR/δ | δ: 0.84-0.87(3H, —CH3), 1.24-1.31 |
| acid (the solvent is deuterated | | (16H, —CH2—), 1.31-1.35(2H, —CH2—), |
| DMSO) | | 1.48-1.55(2H, —CH2—), 2.54-2.61 |
| | | (2H, —CH2—), 3.20(2H, —CH2—), |
| | | 3.63(1H, —NH—), 10.55(—OH) |
| | ¹³C NMR/δ | δ: 14.43(1C, —CH3), 19.53(1C, —CH2—), |
| | | 22.57(1C, —CH2—), 28.79(1C, —CH2—), |
| | | 29.19(1C, —CH2—), 29.28(1C, —CH2—), |
| | | 29,44(1C, —CH2—), 29.49(1C, —CH2—), |
| | | 29,52(1C, —CH2—), 31.77(1C, —CH2—), |
| | | 31.98(1C, —CH2—), 33.45(1C, —CH2—), |
| | | 35.48(1C, —CH2—), 165.53(1C, C═O). |

### Example 4

Flotation of Malachite with 2-(Benzylthio)-Acetohydroxamic Acid:

The concentration of 2-(benzylthio)-acetohydroxamic acid and benzohydroxamic acid was 400 mg/L, the pulp pH was 8, the concentration of a foaming agent (MIBC) was 30 mg/L and the rotation speed was 1650 r/min, malachite with the particle size of −0.076 mm to +0.038 mm was subjected to flotation for 5 minutes respectively. When 2-(benzylthio)-acetohydroxamic acid was used as a collector, 96.26% of malachite can float out, while when benzohydroxamic acid was used as a collector, only 30.88% of malachite can float out.

### Example 5

Flotation of Bauxite with 2-(Benzylthio)-Acetohydroxamic Acid:

The concentration of 2-(benzylthio)-acetohydroxamic acid and benzohydroxamic acid was 150 mg/L, the pulp pH was 8, the concentration of a foaming agent (MIBC) was 30 mg/L and the rotation speed was 1650 r/min, bauxite with the particle size of −0.076 mm to +0.038 mm was subjected to flotation for 5 minutes respectively. When 2-(benzylthio)-acetohydroxamic acid was used as a collector, 95.91% of bauxite can float out, while when benzohydroxamic acid was used as a collector, only 19.88% of bauxite can float out.

### Example 6

Flotation of Wolframite with 2-(Benzylthio)-Acetohydroxamic Acid:

The concentration of 2-(benzylthio)-acetohydroxamic acid and benzohydroxamic acid was 25 mg/L, the pulp pH was 8, the concentration of an activator (Pb²⁺) was 30 mg/L, the concentration of a foaming agent (MIBC) was 30 mg/L and the rotation speed was 1650 r/min, wolframite with the particle size of −0.076 mm to +0.038 mm was subjected to flotation for 5 minutes respectively. The flowchart of the flotation process was as shown in FIG. 10. When 2-(benzylthio)-acetohydroxamic acid was used as a collector, 95.89% of wolframite can float out, while when benzohydroxamic acid was used as a collector, only 46.86% of wolframite can float out.

### Example 7

Flotation of Cassiterite with 2-(Benzylthio)-Acetohydroxamic Acid

The concentration of 2-(benzylthio)-acetohydroxamic acid and benzohydroxamic acid was 400 mg/L, the pulp pH was 8, the concentration of a foaming agent (MIBC) was 30 mg/L and the rotation speed was 1650 r/min, cassiterite with the particle size of −0.076 mm to +0.038 mm was subjected to flotation for 5 minutes respectively. When 2-(benzylthio)-acetohydroxamic acid was used as a collector, 79.41% of cassiterite can float out, while when benzohydroxamic acid was used as a collector, only 42.83% of cassiterite can float out.

What is claimed is:

1. An alkyl thioether ethyl hydroxamic acid beneficiation reagent, wherein the alkyl thioether ethyl hydroxamic acid beneficiation reagent has a structure as shown in formula I, 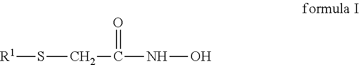
wherein in the formula I, R¹ is one selected from the group consisting of a C₁-C₁₂ alkane group, a C₅-C₁₂ cycloalkyl group, a C₆-C₁₂ aromatic group and a C₁-C₁₂ alkane group and the C₁-C₁₂ alkane group is substituted with at least one substituent.

2. The alkyl thioether ethyl hydroxamic acid beneficiation reagent according to claim 1, wherein R¹ is one selected from the group consisting of methyl, ethyl, propyl, isopropyl, butyl, isobutyl, sec-butyl, tert-butyl, pentyl, isopentyl, sec-pentyl, neopentyl, cyclopentyl, n-hexyl, isohexyl, sec-hexyl, cyclohexyl, heptyl, cycloheptyl, n-octyl, isooctyl, sec-octyl, cyclooctyl, benzyl, phenyl, p-tert-butyl benzyl and dodecyl.

3. The alkyl thioether ethyl hydroxamic acid beneficiation reagent according to claim 2, wherein R¹ is the benzyl or the dodecyl.

4. A preparation method of the alkyl thioether ethyl hydroxamic acid beneficiation reagent according to claim 1, comprising the following steps:
(1) an esterification reaction: subjecting an alkyl thioether acetic acid with a structure as shown in formula II and methanol to the esterification reaction under a catalysis of a concentrated sulfuric acid to obtain a methyl alkyl thioether acetate with a structure as shown in formula III; 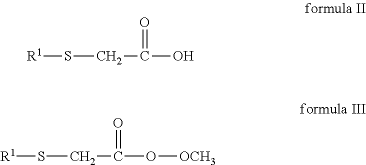
(2) a hydroxamation reaction: subjecting the methyl alkyl thioether acetate with the structure as shown in the formula III, a hydroxylamine and an alkali to the hydroxamation reaction in an aqueous solution to obtain the alkyl thioether ethyl hydroxamic acid beneficiation reagent,

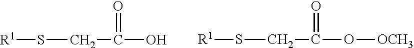

5. The preparation method according to claim 4, wherein in step (1), a temperature of the esterification reaction is 50-100° C., a reaction time is 1-6 hours, a molar ratio of the alkyl thioether acetic acid to the methanol is 1:(1-8), a mass fraction of the concentrated sulfuric acid is 25-50 g/mol, and an added amount of the concentrated sulfuric acid is 2.5-5 g/0.1 mol of the alkyl thioether acetic acid.

6. The preparation method according to claim 4, wherein in step (2), a temperature of the hydroxamation reaction is 10-60° C., a reaction time is 2.5-6 hours, the hydroxylamine is hydroxylamine hydrochloride or hydroxylamine sulfate, the alkali is sodium hydroxide or potassium hydroxide, a molar ratio of the methyl alkyl thioether acetate to the hydroxylamine to the alkali is 1:(1-1.5):(1-1.5), and an amount of water in the aqueous solution is 10-100 mL water/0.1 mol of the methyl alkyl thioether acetate.

7. A method of collecting a flotation of metallic ores, comprising: using the alkyl thioether ethyl hydroxamic acid beneficiation reagent according to claim 1 as a collector for the flotation of the metallic ores.

8. The method according to claim 7, wherein the metallic ores are at least one selected from the group consisting of bauxite, tungsten ores, copper oxide ores and tin ores.

9. The method according to claim 7, wherein a basic process of using the alkyl thioether ethyl hydroxamic acid beneficiation reagent as the collector comprises: (1) floating the metallic ores after grinding to achieve the flotation of the metallic ores; (2) preparing a saline solution of the alkyl thioether ethyl hydroxamic acid as a flotation reagent by mixing the alkyl thioether ethyl hydroxamic acid beneficiation reagent and sodium hydroxide or potassium hydroxide in water; (3) adding hydrochloric acid or the sodium hydroxide during the floating to adjust a pulp pH to 7-9, and adding the saline solution of the alkyl thioether ethyl hydroxamic acid to the flotation of the metallic ores under weakly alkaline conditions to reach a concentration of 25-400 mg/L; and (4) floating useful metallic minerals by a froth flotation method.

10. The preparation method according to claim 4, wherein R¹ is one selected from the group consisting of methyl, ethyl, propyl, isopropyl, butyl, isobutyl, sec-butyl, tert-butyl, pentyl, isopentyl, sec-pentyl, neopentyl, cyclopentyl, n-hexyl, isohexyl, sec-hexyl, cyclohexyl, heptyl, cycloheptyl, n-octyl, isooctyl, sec-octyl, cyclooctyl, benzyl, phenyl, p-tert-butyl benzyl and dodecyl.

11. The preparation method according to claim 10, wherein R¹ is the benzyl or the dodecyl.

12. The method according to claim 7, wherein R¹ is one selected from the group consisting of methyl, ethyl, propyl, isopropyl, butyl, isobutyl, sec-butyl, tert-butyl, pentyl, isopentyl, sec-pentyl, neopentyl, cyclopentyl, n-hexyl, isohexyl, sec-hexyl, cyclohexyl, heptyl, cycloheptyl, n-octyl, isooctyl, sec-octyl, cyclooctyl, benzyl, phenyl, p-tert-butyl benzyl and dodecyl.

13. The method according to claim 12, wherein R¹ is the benzyl or the dodecyl.

14. The method according to claim 8, wherein a basic process of using the alkyl thioether ethyl hydroxamic acid beneficiation reagent as the collector comprises: (1) floating the metallic ores after grinding to achieve the flotation of the metallic ores: (2) preparing a saline solution of the alkyl thioether ethyl hydroxamic acid as a flotation reagent by mixing the alkyl thioether ethyl hydroxamic acid beneficiation reagent and sodium hydroxide or potassium hydroxide in water; (3) adding hydrochloric acid or the sodium hydroxide during the floating to adjust a pulp pH to 7-9, and adding the saline solution of the alkyl thioether ethyl hydroxamic acid to the flotation of the metallic ores under weakly alkaline conditions to reach a concentration of 25-400 mg/L; and (4) floating useful metallic minerals by a froth flotation method.
